(19) United States
(12) Patent Application Publication
(10) Pub. No.: US 2007/0191910 A1
(43) Pub. Date: Aug. 16, 2007

(54) Visual prosthesis

(76) Inventor: Qiushi Ren, Irvine, CA (US)

Correspondence Address: NEIL K. NYDEGGER;NYDEGGER & ASSOCIATES, 348 Olive Street, San Diego, CA, 92103, US

(21) Appl. No.: 11/356,367

(22) Filed: Feb. 16, 2006

(51) Int. Cl. A61N 1/05; A61F 9/00

(52) U.S. Cl. 607/54

(57) ABSTRACT

A prosthesis for viewing an object includes a CMOS camera that is mounted on an eye lens of a patient to create a visual image. A process controller then creates an encoded signal of the visual image that has specific intensity and position information for respective pixels. Next, this encoded signal is transmitted to a stimulating unit implanted in the cranium, where it is decoded to create an electrical stimulation signal in accordance with a predetermined data protocol. The electrical stimulation signal is then passed to an electrode array that is coupled to an optic nerve of the patient, to thereby generate phosphenes in the brain for a sensation of visual perception of the object.

### FIELD OF THE INVENTION

The present invention pertains generally to visual prosthetic devices for blind persons and to the related surgical technique for implanting the device. More particularly, the present invention pertains to devices and methods for stimulating nerve fibers in the optic nerve to generate phosphenes for a sensation of visual perception. The present invention is particularly, but not exclusively, useful as a device that electronically bypasses the retina to generate a visual image of an object, and then uses image-based signals to directly stimulate nerve fibers in the optic nerve.

### BACKGROUND OF THE INVENTION

With a normal eye, the visual perception of an object results when light is anatomically converted into impulse signals that will neurologically stimulate the brain. In overview, this conversion essentially involves three separate, but interrelated, functions. First, light from whatever is being viewed needs to be focused in order to create an image. In an eye, this focusing is done by the eye's lens and its associated anatomy. Secondly, the focused image is converted into neurological impulse signals by the retina (fundus). In this function, the retina actually works together with a complex nerve structure that is associated with the retina. Finally, as the third defined function, the neurological impulse signals are transferred from the retina to a portion of the central nervous system in the brain. This transfer is accomplished by the optic nerve. When any part of this anatomical optical system becomes inoperable or dysfunctional, for whatever reason, intervention becomes necessary in order to restore sight. Fortunately, some situations yield to modern medical treatments. Others, however, do not.

Of particular interest here is the situation wherein the retina alone becomes essentially ineffective and unusable for its intended purpose. Some diseases of the eye, such as retinitis pigmentosa and macular degeneration, present just such a circumstance. In these cases, other parts of the optical pathway, and most importantly the optic nerve, remain healthy and functional. How the optic nerve can be most effectively used in such a circumstance becomes a question of great importance.

Anatomically, the optic nerve is a cordlike structure that is composed of numerous nerve fibers. More specifically, the nerve fibers are grouped into several individual bundles that are each surrounded by a sheet of connective tissue (perineurium). Together, the bundle of nerve fibers and its perineurium are referred to as a fasciculus. The fasciculi are then bound together by a thick layer of connective tissue (epineurium) to form the optic nerve. Of importance here is the fact that nerve fibers of the optic nerve are used to transfer bio-electrical impulses from the retina to the brain to give a sensation of light. By definition, a phosphene is a sensation of light that is caused by excitation of the neurological tissues along the visual pathway by mechanical or electrical means, rather than by light.

Heretofore, attempts to provide visual perceptions for blind people have included the use of devices that are mounted directly onto the retina. Typically, these devices incorporate electrodes that stimulate neurons (ganglions) of the retina (see U.S. Pat. No. 4,628,933 and U.S. Pat. No. 5,109,844). These examples, however, rely on a direct electrical stimulation of the retina, and do not suggest how a diseased retina can be effectively bypassed, and still generate a visual sensation for the patient.

In light of the above, it is an object of the present invention to provide a system, and a method for its use, that generates phosphenes by stimulating the optic nerve to give a patient a visual perception of an object. Another object of the present invention is to provide a system and method that can effectively bypass a diseased or dysfunctional retina, and still give a patient a visual perception of an object. Yet another object of the present invention is to provide a visual prosthesis, and a method for its use, that is simple to use, relatively easy to manufacture, and comparatively cost effective.

### SUMMARY OF THE INVENTION

In accordance with the present invention, a visual prosthesis for use by an otherwise blind patient, directly stimulates the optic nerve of the patient while effectively bypassing the retina. Importantly, an electrical image of an object being viewed is created. This electrical image is then used to create impulse signals that are representative of the image. These impulse signals are then arranged according to a predetermined data protocol, and are electronically passed directly to nerve fibers in the optic nerve. There, in the optic nerve, phosphenes are generated for a sensation of visual perception of the object.

In detail, the prosthesis of the present invention includes an implantable intraocular micro-camera for creating an image of the object being viewed. Preferably, the camera is a CMOS-technology-based camera, and it is situated in the posterior lens chamber of an eye of the patient. A wide-angle auto-focus lens is mounted on the micro-camera to focus light from the object onto the camera.

For one embodiment of the present invention, the front end of the micro-camera includes a disk-like solar panel that performs two functions. First, the solar panel provides energy to the micro-camera and its functional circuitry. Second, it provides mechanical support to the micro-camera device as it is situated on top of the iris of an eye of the patient. In other embodiments of the present invention, the micro-camera can be battery powered or RF powered (wireless). In each embodiment, the lens and a chamber interact to hermetically seal the micro-camera inside the chamber. As intended for the present invention, the visual image that is created by the camera comprises a discreet number of pixels, wherein each pixel is characterized by an intensity and a predetermined position that is representative of the visual image being viewed.

A process controller for creating an encoded visual image signal is positioned to electronically receive the visual image from the camera. As envisioned for the present invention, the process controller may, or may not, be extracorporeal. In either case, the process controller encodes the visual image to create an encoded visual image signal that includes a plurality of electrical impulses. Specifically, each electrical impulse in the encoded visual image signal includes information that characterizes the intensity and position of a respective pixel. If the process controller is extracorporeal, the encoded visual image signal is modulated onto an electromagnetic radio frequency carrier. This modulation creates a transmit signal that is transmitted to a stimulating unit implanted in the cranium of the patient.

The intracranially implanted stimulating unit is used in the present invention for receiving the encoded visual image signal. This is done either directly from the process controller, if the process controller is also implanted, or with the transmit signal (i.e. a modulated encoded visual signal), if the process controller is extracorporeal. In either case, the received signal is then decoded in the stimulating unit to create an electrical stimulation signal. For the present invention it is important that this decoding of the encoded visual image signal (i.e. the creation of the electrical stimulation signal) be accomplished in accordance with a predetermined data protocol. As recognized and appreciated by the present invention, this data protocol is patient specific and will differ from one patient to another. Consequently, the predetermined data protocol that is used to decode the encoded visual image signal is customized for the particular patient. This is done in training sessions where the data protocol is established so that each pixel address from the visual image will correspond with a specific nerve fiber in the optic nerve. Collectively, the nerve fibers in the optic nerve can then give the patient a sensation of visual perception of the object being viewed. To do this, the electrical stimulation signal is passed to the nerve fibers of the optic nerve by an electrode array.

For the present invention the electrode array is coupled directly to an optic nerve of the patient. It is also electrically connected with the stimulating unit to receive the electrical stimulation signal. Structurally, the electrode array includes a base member having a plurality of probes that are mounted on the base member for embedment into the optic nerve of the patient. At least one electrode is mounted on each probe, and each electrode on the probe is in electrical communication with the stimulating unit. Specifically, each electrode receives a portion of the electrical stimulation signal. Recall the predetermined data protocol. In accordance with this protocol, a phospene is generated by an electrode as the electrode receives its portion of the electrical stimulation signal. Importantly, according to the data protocol, this phosphene will correspond with the appropriate pixel in the visual image of the object. There are, of course, many such electrodes. Collectively, the result is an accurate visual perception of the object.

In greater structural detail, the electrode array for the present invention may include a plurality of probes that are aligned in rows on the base member. Further, there may be a plurality of base members in the array. Regardless how many base members and how many probes there may be, each probe will preferably have a length that is in a range between five hundred microns and five millimeters (500 μm-5 mm). Also, the probes can be spaced from adjacent probes by a distance that is in a range between fifty microns and five millimeters 50 μm-5 mm).

In another embodiment of the stimulating electrode array, a parylene or polymer based, paper-like material can be used as a base member for an electrode array. The thickness of such base members is in a range between 1 micron to 2 millimeters (1 μm-2 mm). In this embodiment, the geometrical arrangement of the probes on each base member of the electrode array is stair-like, with each probe preferably having a length that is in a range between five hundred microns and five millimeters (500 μm-5 mm). Also, the probes can be spaced both horizontally and vertically from adjacent probes on the same base member by a distance that is in a range between ten microns and five millimeters (10 μm-5 mm). Specifically, such an arrangement is provided in order to avoid stimulating the same optic nerve fiber or fibers by different probes, and thereby reducing the overall efficiency of the electrode array.

Two different methods are envisioned for inserting an electrical electrode array into its operational position. In one (i.e. the paper-like array), the optic nerve will first be dissected to create an opening along the axis of the optic nerve. The paper-like stimulating electrode array will then be inserted into the opening with the cable sitting outside the optic nerve for connection to the stimulating unit. In this manner, a plurality of such electrode arrays can be inserted around the optic nerve of the patient to increase the resolution of the vision. In another (i.e. the pin-like electrode), a plurality of pin-like arrays are pushed into the optic nerve. The pin-like array penetrates into the optical nerve and each electrode on each pin will contact fibers of the optical nerve.

In other aspects of the present invention, the stimulating unit may include an error detection circuit that will stop the creation of the electrical stimulation signal whenever the predetermined data protocol is not being followed. Further, the electrical stimulation signal is preferably biphasic so that no net electrical charge is delivered to tissue in the cranium.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a schematic view of an embodiment of a visual prosthesis for the present invention;

FIG. 1B is a schematic view of an alternate embodiment of the visual prosthesis for the present invention;

FIG. 2 is a schematic view of an implantable micro-camera that can be either battery powered, solar powered, or RF powered (wireless);

FIG. 3 is a cross-sectional view of an optic nerve with a surgically implanted electrode array;

FIG. 4 is a cross-sectional view of an electrode array as would be seen along the line 4-4 in FIG. 1A when the array is open and disconnected from an optic nerve;

FIG. 5A is a cross-sectional view of an electrode probe as seen along the line 5-5 in FIG. 4;

FIG. 5B is a schematic view of an alternative embodiment of a stimulating electrode array;

FIG. 6A is a cross-sectional view of an electrode array as seen in FIG. 4, when the array is closed and embedded into an optic nerve;

FIG. 6B is a perspective view of the stimulating electrode array as seen in FIG. 5B when inserted and embedded into the optic nerve;

FIG. 7 is a visual image of an object;

FIG. 8 is a row of pixels that are characteristic of a line on an object such as shown in FIG. 7; and

FIG. 9 is a schematic presentation of a data protocol as used for the present invention.

### DESCRIPTION OF THE PREFERRED EMBODIMENTS

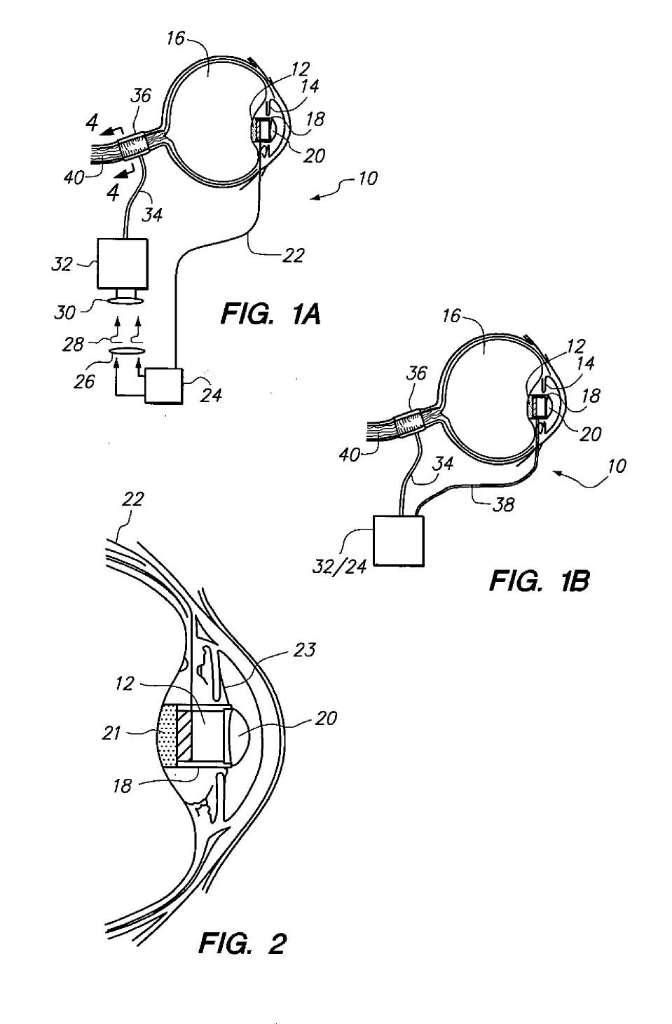

Referring initially to FIG. 1A, a visual prosthesis in accordance with the present invention is shown and is generally designated 10. As shown, the prosthesis 10 includes an intraocular (CMOS) camera 12 that is mounted on the lens 14 in an eye 16 of a patient. Further, as also shown in FIG. 1A, the camera 12 is mounted in a chamber 18 along with a wide angle lens 20. Together, the chamber 18 and lens 20 provide a hermetic seal for the camera 12. Preferably, the camera 12 is a CMOS camera having a sensor area 3.37 mm×2.54 mm, with pixel sizes of 2.0 μm×2.0 μm and an output of 25 frames per second at 128×128 pixel resolution. Other, similar type cameras with different pixel size and resolution, however, could be used as well. As envisioned for the present invention, the various types of CMOS cameras that can be used as the camera 12 will vary primarily in the type of power supply that is used. Referring now to FIG. 2 it will be seen that the camera 12 typically includes a power pack 21 that provides operational power for the camera 12. With this combination, at least three different sources of power are envisioned for camera 12. For one, power can be supplied to the camera 12 via a transcutaneous connection. In particular, such a connection will likely be with an extracorporeal voltage source, such as a battery (not shown). For another, the camera 12 may include an energy transfer unit 23 that is effectively mounted on the chamber 18. In one embodiment, this energy transfer unit 23 can be a solar panel that will transfer energy to the power pack 21 for subsequent use. In another embodiment, the energy transfer unit 23 may be an RF antenna that will pass energy via a wireless connection to the power pack 21. In any event, the present invention envisions several different type devices for powering the camera 12.

Referring back to FIG. 1A, it is seen that a transcutaneous electronic line 22 connects the intraocular camera 12 with a process controller 24. For the embodiment of the visual prosthesis 10 shown in FIG. 1A, the process controller 24 is envisioned as being extracorporeal. As shown, the process controller 24 includes an antenna 26 that radiates a modulated, radio frequency, transmit signal 28. For the visual prosthesis 10, the transmit signal 28 is received by an antenna 30 of a stimulating unit 32. As envisioned for the visual prosthesis 10, the stimulating unit 32 and its antenna 30 can be intracranially implanted. Additional components for completing the electronic aspects of the visual prosthesis 10 include a cable 34 that electronically connects the stimulating unit 32 with an electrode array 36.

In an alternate embodiment of the present invention, a visual prosthesis as shown in FIG. 1B and generally designated 10′ essentially includes the same electronic components as disclosed above for the visual prosthesis 10. For the visual prosthesis 10′, however, the process controller 24 is not extracorporeal. Instead, it is electronically combined with the stimulating unit 32 and, in this combination, the process controller 24 is intracranially implanted with the stimulating unit 32. For this embodiment, a connecting line 38 is implanted under the skin to directly connect the camera 12 with the stimulating unit/process controller 32/24. Thus, there is no need to generate a transmit signal 28. For both embodiments of the present invention (i.e. visual prosthesis 10 and visual prosthesis 10′), the electrode array 36 is connected to the optic nerve 40 of a patient (not shown).

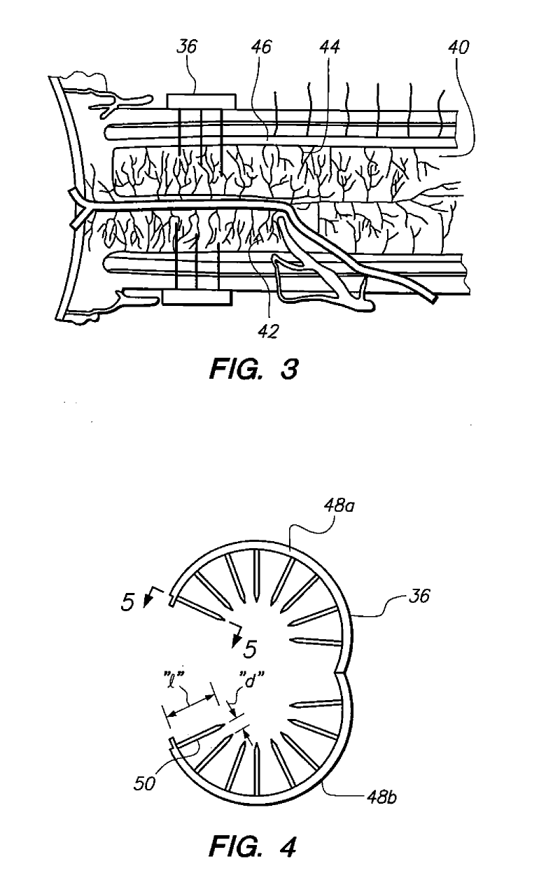

The microanatomy of the optic nerve 40 is important for the present invention and can be best appreciated with reference to FIG. 3. There it will be seen that blood vessels 42 are located at the center of the optic nerve 40. Surrounding these blood vessels 42 is a plurality of nerve fiber groups 44, each of which is surrounded by a sheet of connective tissue (i.e. perineurium). Together, the nerve fiber group 44, and its surround perineurium as known as a fasciculus. The fasciculi are then bound and held together by a thick layer of connective tissue 46 (i.e. epineurium) to form the optic nerve 40. As indicated above, how an electrode array 36 (or a similar device employed for the same purpose) interacts with the optic nerve 40 is an aspect of great importance for the visual prosthesis 10 (10′) of the present invention.

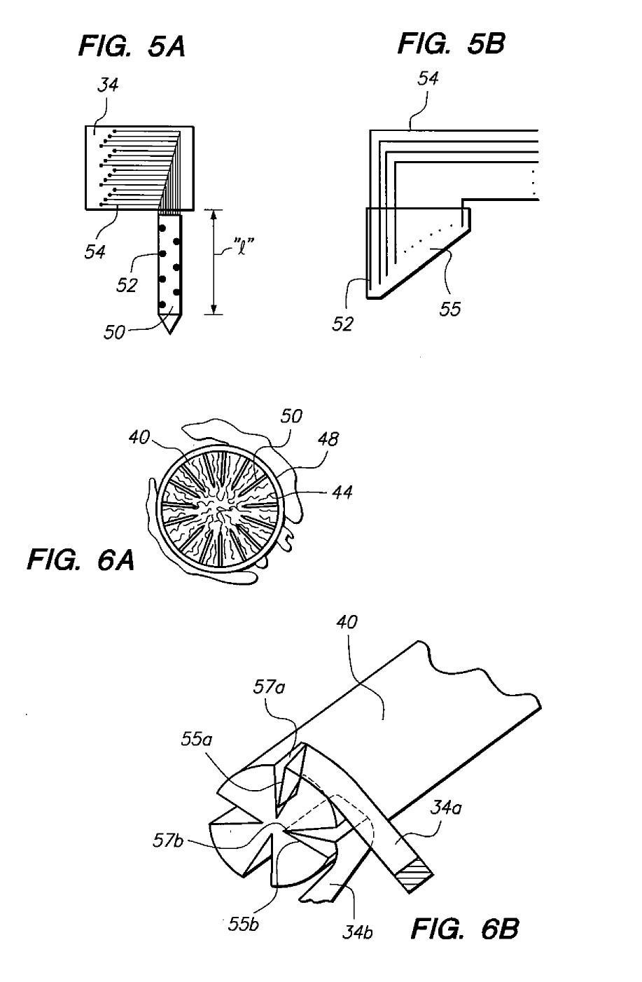

Referring now to FIG. 4, an electrode array 36 for use in the present invention is shown to include at least one base member 48 (the base members 48a and 48b are exemplary). Further, at least one probe 50 is mounted on the base member 48. Preferably, however, there will be a plethora of probes 50 mounted on each base member 48. Also, it is envisioned that the probes 50 will be aligned in rows on the base member 48 and that each probe 50 will have a length “l” that is in a range between five hundred microns and five millimeters (500 μm-5 mm). Also, it is envisioned that each probe 50 will be spaced from an adjacent probe 50 by a distance “d” that is in a range between ten microns and five millimeters (10 μm-5 mm). As best seen in FIG. 5A, the probes 50 will include at least one electrode 52. Most likely, however, there will be a plethora of electrodes 52. The electrode 52 that is shown for the probe 50 in FIG. 5A is only exemplary and its diameter is in a range between ten microns and one millimeter (10 μm and 1 mm). Importantly, each electrode 52 will be electronically connected with a respective line 54, and each line 54 will become part of the cable 34 that electronically interconnects the electrode array 36 with the stimulating unit/process controller 32/24. As perhaps best appreciated with reference to FIG. 6A, and with cross reference to FIG. 1A or FIG. 1B, the probes 50 of electrode array 36 are embedded into the optic nerve 40 to establish electrical contacts between the electrodes 52 and the nerve fiber groups 44 (see FIG. 3).

In an alternate embodiment of a stimulating electrode array for the prosthesis 10 of the present invention, shown in FIG. 5B, a flat, paper-like base member 55 is provided for the electrodes 52. In detail, for this embodiment, the base member 55 is preferably a parylene or polymer based paper material on which the electrodes 52 are mounted in a manner well known in the pertinent art, such as by bonding or printing. Preferably, the thickness of the base member 55 will be in a range between 1 micron and two millimeters (1 μm-2 mm), and the geometrical arrangement of the electrodes 52 will be generally stair-stepped. Again, as with the embodiment for the electrode array 36 disclosed above, the separation distance between adjacent electrodes 52 is in a range between ten microns and five millimeters (10 μm-5 mm) in order to prevent two different electrodes 52 from stimulating the same nerves in a nerve fiber group 44.

In accordance with the present invention, the implementation of stimulating electrode arrays that incorporate flat, paper-like base members 55 will be best appreciated with reference to FIG. 6B. There, it will be seen that a plurality of openings 57 (e.g. 57a and 57b), is axially incised along the length of the optic nerve 40. Then, for example, once openings 57a and 57b have been prepared, the base member 55a and 55b of respective stimulating electrode arrays are positioned therein for the purpose of establishing electrical contact between electrodes 52 on the base members 55a and 55b, and nerves in various nerve fiber groups 44 of the optic nerve 40. As disclosed above, the electrodes 52 are in electrical contact with the stimulating unit 32 via cables 34. As envisioned for the present invention, the electrodes 52 on base member 55a and base member 55b will be in electrical contact with the stimulating unit 32 via respective cables 34a and 34b.

### Operation

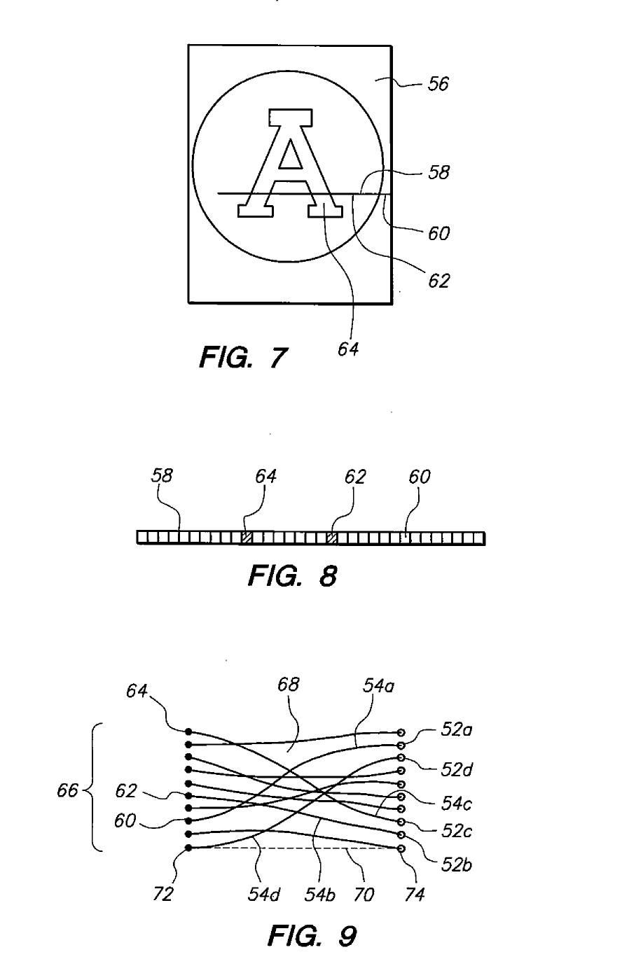

For the operation of the visual prosthesis 10, the intraocular camera 12 is aimed by moving the eye 16. Thus, the camera 12 is able to receive light reflected from an object 56 (e.g. see FIG. 7). As will be appreciated by the skilled artisan, the camera 12 effectively digitizes the light that is reflected from the object 56 to create an image. This image comprises a plurality of discreet pixels. More specifically, each pixel in the visual image has a location and a light intensity that is characteristic of the object 56 that is being imaged. For example, consider the line 58 that is shown on the object 56 in FIG. 7, and cross-reference this with FIG. 8. When doing so, it will be seen that along the line 58 there is a pixel 60 at a specific position on the line 58 that is characterized by light having a relatively high intensity. Another pixel 62, however, at another position on the line 58, will comprise light having a relatively medium intensity. Still another pixel 64 will comprise low intensity light. As indicated, each of the pixels 60, 62 and 64 has a respective position on the line 58, as well as an intensity, that is indicative of a corresponding location on the object 56. All of the pixels shown here are, of course, only exemplary. Also, it will be appreciated there are many pixels in each line 58, and that many such lines 58 are required to reproduce the object 56 in its entirety.

In a manner well known in the pertinent art, each pixel (e.g. pixels 60, 62 and 64) in a. visual image of the object 56 is converted into an electrical impulse. A plurality of the electrical impulses is then used by the process controller 24 to create an encoded visual image signal. Once it is created, this encoded visual image signal is transferred to the stimulating unit 32. For the embodiment of the visual prosthesis 10 that is shown in FIG. 1A, this transfer requires the encoded visual image signal be converted to a transmit signal 28. Preferably, the conversion to the transmit signal 28 is accomplished by modulating a radio frequency carrier with the information contained in the encoded visual image signal. As indicated above, the transmit signal 28 is transmitted from antenna 26 to antenna 30, and is then received by the stimulating unit 32. For the embodiment of the visual prosthesis 10′ that is shown in FIG. 1B, the process controller 24 and stimulating unit 32 are effectively combined, and both are implanted together in the cranium of the patient. This obviates the need to generate the transmit signal 28. In either case, however, the encoded visual image signal is decoded at the stimulating unit 32 to create an electrical stimulation signal 66 in accordance with a predetermined data protocol 68 (see FIG. 9).

As represented in FIG. 9, a predetermined data protocol 68 is essentially, an electrical diagram that uses the electrical stimulation signal 66 to stimulate nerve fibers in a nerve fiber group 44 with an appropriate electrode 52. The consequence of this is the generation of a phosphene in the optic nerve 40 for a sensation of visual perception of the object 56. Anatomically, however, there is no direct correlation between a pixel from the object 56 that is electronically created for the stimulation signal 66 (e.g. pixel 72), and a nerve fiber group 44 that needs to be stimulated by an electrode 74 for a visual sensation of an object 56. Such a direct connection 70 (see the dashed line in FIG. 9) would be convenient, but not realistic. Instead, all patients are different. Consequently, an alignment of pixels, such as pixels 60, 62 and 64 from an object 56, will inevitably encounter an unpredictable arrangement of nerve fiber groups 44. Thus, the electrodes 52 need to be electronically rearranged in order to properly generate phosphenes in the optic nerve 40. For example, the pixels 60, 62 and 64 from object 56 are physically aligned. And, this alignment is followed in the stimulation signal 66. In order for the patient to use this stimulation signal 66 to achieve a visual sensation of the object 56, however, the nerve fiber groups 44 of optic nerve 40 will need to be stimulated in a different order. Indeed, for the example shown in FIG. 9, it can happen that a proper order for stimulating electrodes to create a sensation for a visual image of the object 56 might well be 52b, 52c and then 52a via respective connections indicated by lines 54b, 54c, and 54a. For the present invention, this rearrangement of electrode stimulation is done in accordance with the data protocol 68. And, the data protocol 68 is established by training the patient until the electrical stimulation signal 66 reliably stimulates nerve fiber groups 44 in the optic nerve 40. Importantly, these stimulations need to faithfully recreate a sensation of visual perception of the object 56 for the patient.

While the particular Visual Prosthesis as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A visual prosthesis for use by a patient in viewing an object which comprises:
an intraocular camera situated in the posterior lens chamber on an eye of the patient for creating a visual image of the object, wherein the visual image comprises a discreet number of pixels, with each pixel having an intensity and a predetermined position in the visual image;
a process controller for creating an encoded visual image signal, wherein the encoded visual image signal comprises a plurality of electrical impulses with each electrical impulse including characteristics of the intensity and position of a respective pixel;
a stimulating unit for receiving and decoding the encoded visual signal to create an electrical stimulation signal in accordance with a predetermined data protocol; and
an electrode array coupled to an optic nerve of the patient and electrically connected with said stimulating unit for receiving and using the electrical stimulation signal to generate phosphenes in the optic nerve for a sensation of visual perception of the object.

2. A prosthesis as recited in claim 1 wherein said camera is a CMOS camera and said prosthesis further comprises:
a cylindrical chamber for holding the CMOS camera therein; and
a wide-angle lens mounted on said chamber for directing a view of the object to the CMOS camera, wherein said lens and said chamber interact to hermetically seal the CMOS camera in said chamber.

3. A prosthesis as recited in claim 1 wherein said process controller comprises:
a means for generating an electromagnetic radio frequency carrier; and
a means for modulating the encoded visual image signal onto the carrier to create a transmit signal.

4. A prosthesis as recited in claim 3 further comprising:
a first antenna mounted on said process controller for transmitting the transmit signal; and
a second antenna mounted on said stimulating unit for receiving the transmit signal.

5. A prosthesis as recited in claim 4 wherein said process controller is extracorporeal and said stimulating unit is intracranially implanted.

6. A prosthesis as recited in claim 1 wherein said electrode array comprises:
a base member;
a plurality of probes mounted on said base member for embedment into the optic nerve of the patient; and
at least one electrode mounted on each said probe, wherein each electrode is in electrical communication with said stimulating unit for receiving a portion of the electrical stimulation signal therefrom in accordance with the predetermined data protocol to correspond the phospene generated by the electrode with the appropriate pixel in the visual image of the object for an accurate visual perception of the object.

7. A prosthesis as recited in claim 6 wherein a plurality of probes are aligned in a row on said base member, with each probe having a length in a range between five hundred microns and five millimeters (500 μm-5 mm) and wherein each probe is spaced from an adjacent probe by a distance in a range between ten microns and five millimeters (10 μm-5 mm).

8. A prosthesis as recited in claim 7 further comprising a plurality of rows of probes on said base member.

9. A prosthesis as recited in claim 6 further comprising a plurality of base members.

10. A prosthesis as recited in claim 1 wherein said stimulating unit comprises an error detection circuit for stopping creation of the electrical stimulation signal whenever the predetermined data protocol is not being followed.

11. A prosthesis as recited in claim 1 wherein the electrical stimulation signal is biphasic.

12. A device for generating phosphenes for sensing a visual perception of an object which comprises:
a means for creating a visual image of the object, wherein the visual image comprises a discreet number of pixels, with each pixel having an intensity and a predetermined position in the visual image;
a means for encoding the visual image signal, wherein the encoded visual image signal comprises a plurality of electrical impulses with each electrical impulse including characteristics of the intensity and position of a respective pixel;
a means for transmitting the encoded visual image to an intracranially implanted stimulation unit for decoding the encoded visual signal to create an electrical stimulation signal in accordance with a predetermined data protocol; and
an electrode array coupled to an optic nerve of the patient and electrically connected with said stimulating unit for receiving and using the electrical stimulation signal to generate phosphenes in the optic nerve for a sensation of visual perception of the object.

13. A device as recited in claim 12 wherein said visual image creating means is a CMOS camera having a sensor area 3.37 mm×2.54 mm, with pixel sizes of 2.0 μm×2.0 μm and an output of 25 frames per second at 128×128 pixel resolution.

14. A device as recited in claim 13 further comprising:
a cylindrical chamber for holding the CMOS camera therein; and
a wide-angle lens mounted on said chamber for directing a view of the object to the CMOS camera, wherein said lens and said chamber interact to hermetically seal the CMOS camera in said chamber.

15. A device as recited in claim 14 wherein said process controller comprises:
a means for generating an electromagnetic radio frequency carrier;
a means for modulating the encoded visual image signal onto the carrier to create a transmit signal; and
a first antenna for transmitting the transmit signal to a second antenna mounted on said stimulating unit for receiving the transmit signal.

16. A device as recited in claim 12 wherein said electrode array comprises:
a base member;
a plurality of probes mounted on said base member for embedment into the optic nerve of the patient; and
at least one electrode mounted on each said probe, wherein each electrode is in electrical communication with said stimulating unit for receiving a portion of the electrical stimulation signal therefrom in accordance with the predetermined data protocol to correspond the phospene generated by the electrode with the appropriate pixel in the visual image of the object for an accurate visual perception of the object.

17. A method for generating phosphenes in the optic nerve of a patient for sensing a visual perception of an object, said method comprising the steps of:
positioning a camera to create a visual image of the object, wherein the visual image comprises a discreet number of pixels, with each pixel having an intensity and a predetermined position in the visual image;
transferring the visual image to a process controller for creating an encoded visual image signal, wherein the encoded visual image signal comprises a plurality of electrical impulses with each electrical impulse including characteristics of the intensity and position of a respective pixel;
transmitting the encoded visual image from the process controller to a stimulating unit for decoding in the stimulating unit to create an electrical stimulation signal in accordance with a predetermined data protocol; and
passing the electrical stimulation signal to an electrode array coupled to the optic nerve to generate phosphenes in the optic nerve for a sensation of visual perception of the object by the patient.

18. A method as recited in claim 17 wherein said camera is a CMOS camera and said method further comprises the steps of:
placing a cylindrical chamber in the lens of the patient for holding the CMOS camera in the chamber; and
mounting a wide-angle lens on the chamber for directing a view of the object to the CMOS camera, wherein said lens and said chamber interact to hermetically seal the CMOS camera in said chamber.

19. A method as recited in claim 18 wherein said process controller comprises:
a means for generating an electromagnetic radio frequency carrier;
a means for modulating the encoded visual image signal onto the carrier to create a transmit signal; and
a first antenna for transmitting the transmit signal to a second antenna mounted on said stimulating unit for receiving the transmit signal.

20. A method as recited in claim 17 wherein said electrode array comprises:
a base member;
a plurality of probes mounted on said base member for embedment into the optic nerve of the patient; and
at least one electrode mounted on each said probe, wherein each electrode is in electrical communication with said stimulating unit for receiving a portion of the electrical stimulation signal therefrom in accordance with the predetermined data protocol to correspond the phospene generated by the electrode with the appropriate pixel in the visual image of the object for an accurate visual perception of the object.
